(19) United States
(12) Patent Application Publication
(10) Pub. No.: US 2019/0102884 A1
(43) Pub. Date: Apr. 4, 2019

(54) IMAGE SIMULATION SYSTEM TO COVISUALIZE ELECTRODES AND CORTICAL VESSELS ON 3D BRAIN AND ITS METHOD

(71) Applicant: CHUNG SHAN MEDICAL UNIVERSITY HOSPITAL, Taichung City (TW)

(72) Inventors: YUE-LOONG HSIN, Taichung City (TW); SYU-JYUN PENG, Zhubei City (TW); CHENG-SIU CHANG, Taichung City (TW); FU-YUAN SHIH, Kaohsiung City (TW)

(21) Appl. No.: 15/826,652

(22) Filed: Nov. 29, 2017

(30) Foreign Application Priority Data
Oct. 2, 2017 (TW) 106134082

(51) Int. Cl. G06T 7/00; G06F 17/50; G16H 30/40

(52) U.S. Cl. CPC G06T 7/0016; A61B 5/055; G16H 30/40; G06F 17/5009

(57) ABSTRACT

A method comprises steps of capturing first images for displaying a three-dimensional brain structure, capturing second images for displaying blood vessels of the brain and capturing third images displaying implanted electrodes in the brain. These second images are set as standards to respectively adjust and align the first images and the third images. Contrast of the blood vessels is enhanced and the blood vessels are colored with a first color. Contrast of the electrodes is enhanced and the electrodes are colored with a second color. The aligned first images, the colored second images and the colored third images are integrated to obtain integrated viewable information of a brain, (intracranial) electrodes and blood vessels for medical reference. So, spatial positions of implanted electrodes and blood vessels of brains can be confirmed before conducting a medical surgery. And, the physicians can avoid bleeding problem due to accidental touch on blood vessels.

### BACKGROUND OF THE INVENTION

### 1. Field of the Invention

The present invention is related to an image simulation system and method for covisualizing electrodes and cortical vessels on a three dimensional (3D) brain structure diagram, and more particularly to an image simulation system and method for covisualizing electrodes and cortical vessels on a 3D brain structure diagram having advantages that spatial positions of implanted electrodes and blood vessels of brains can be confirmed in advance at the time of medical surgery, and that physicians can avoid bleeding problem due to accidental touch on blood vessels.

### 2. The Related Arts

Although a conventional method disclosed by Taiwan patent No. 1318874 titled “A Method to Construct a Three-Dimensional Image of Human Tissues” discloses a method of constructing a three-dimensional structure of brains, no technique of displaying electrodes and blood vessels in the brains is provided at the same time.

In addition, although a method disclosed by Taiwan patent No. 201225922 titled “An Angiography Method for Visualization of Trans-Bone Blood Vessels” includes angiography using contrast media, no technique of electrode integration and electrode images is provided in the above mentioned patent.

Especially for patients with epilepsy, when electrodes have been intracranially implanted, traditional ways cannot simultaneously learn relative position information of brain structures, blood vessel distribution and electrode positions.

In view of the above, it is necessary to develop a technology that can solve the above disadvantages of conventional techniques.

### SUMMARY OF THE INVENTION

An object of the present invention is to provide an image simulation system and method for covisualizing electrodes and cortical vessels on a three dimensional (3D) brain structure diagram. Advantages of the present invention include that spatial positions of implanted electrodes and blood vessels of brains can be confirmed in advance at the time of medical surgery, and that physicians can avoid bleeding due to accidental touch on blood vessels, and so on. In particular, drawbacks of conventional technology such that relative positions of (intracranial) electrodes and blood vessels in brains cannot be known at the same time in view of conventional image information are problems intended to be solved by the present invention.

Technical solutions to solve the above mentioned drawbacks are to provide an image simulation system and method for covisualizing electrodes and cortical vessels on a three dimensional (3D) brain structure diagram. An image simulation system in accordance with the present invention comprises the following.

A first image capturing device is used to capture a plurality of first images of a brain in advance. The plurality of first images are completely used to display a three-dimensional structure of the brain.

A second image capturing device is used to capture a plurality of second images of the brain in advance. The plurality of second images are completely used to display blood vessels of the brain.

A third image capturing device is used to capture a plurality of third images of the brain after the brain is implanted with at least one electrode.

The plurality of third images are completely used to display the electrode in the brain.

A processing device is data-communicably connected to the first image capturing device, the second image capturing device and the third image capturing device. The plurality of second images are retrieved and set as standards, and a plurality of first images are correspondingly retrieved and aligned with the plurality of retrieved second images in order to generate a plurality of aligned first images. Contrast between the blood vessel and its adjacent non-vessel portions shown in the plurality of retrieved second images is firstly enhanced via image processing in order to visualize the blood vessel. The blood vessel is further colored with a first color in order to visualize a location of the blood vessel at the brain in the plurality of retrieved second images. In addition, the plurality of retrieved second images are set as standards, and a plurality of third images are correspondingly retrieved and aligned with the plurality of retrieved second images in order to generate a plurality of aligned third images. Contrast between the electrode and its adjacent non-electrode portions shown in the plurality of aligned third images are firstly enhanced via image processing in order to visualize the electrode. The electrode is further colored with a second color in order to visualize a location of the electrode at the brain in the plurality of aligned third images. Finally, the plurality of aligned first images, the plurality of colored second images and the plurality of colored third images are integrated to obtain integrated viewable information of a brain, (intracranial) electrodes and blood vessels for medical reference.

An image simulation method in accordance with the present invention comprises the following steps.

1. A step of capturing a plurality of first Images.

2. A step of capturing a plurality of second Images.

3. A step of capturing a plurality of third Images.

4. A step of image alignment.

5. A step of coloring.

6. A step of integrating.

The above objects and advantages of the present invention can be easily understood in depth from the following detailed descriptions of preferred embodiments of the present invention and accompanying drawings.

The present invention is further illustrated and explained in details by the following preferred embodiments of the present invention and accompanying drawings as follows.

### BRIEF DESCRIPTION OF THE DRAWINGS

The present invention will become more readily apparent to those ordinarily skilled in the art after reviewing the following detailed description and accompanying drawings, in which:

FIG. 1A is a schematic block diagram of an image simulation system in accordance with the present invention.

FIG. 1B is a schematic block diagram of an image capturing process of FIG. 1A.

FIG. 2 is a schematic perspective view showing a plurality of cross-sectional images taken from a brain in accordance with the present invention.

FIG. 3 is a schematic perspective view showing an electrode (a needle-insertion style) disposed in a brain in accordance with the present invention.

FIG. 4 is a schematic cross-sectional view of FIG. 3 in accordance with the present invention.

FIG. 5 is a schematic perspective view showing an electrode in accordance with an embodiment of the present invention being of a two-dimensional matrix film type.

FIG. 6 is a schematic block diagram of an image simulation method system in accordance with the present invention.

FIGS. 7A, 7B, 7C and 7D are schematic diagrams of a plurality of first image at the 146th to 149th layers of the brain in accordance with the present invention before image alignment.

FIGS. 7E, 7F, 7G and 7H are schematic diagrams respectively showing image diagrams of FIGS. 7A, 7B, 7C and 7D after image alignment in accordance with the present invention.

FIGS. 8A, 8B, 8C and 8D are schematic diagrams of a plurality of second image at the 146th to 149th layers of the brain in accordance with the present invention before coloring.

FIGS. 8E, 8F, 8G and 8H are schematic diagrams respectively showing image diagrams of FIGS. 8A, 8B, 8C and 8D after being colored (visualizing blood vessels) in accordance with the present invention.

FIG. 8I is a schematic enlarged diagram of FIG. 8E in accordance with the present invention.

FIG. 8J is a schematic diagram of FIG. 8I showing visualized blood vessels in accordance with the present invention.

FIGS. 9A, 9B, 9C and 9D are schematic diagrams of a plurality of third image at the 146th to 149th layers of the brain in accordance with the present invention before image alignment.

FIGS. 9E, 9F, 9G and 9H are schematic diagrams respectively showing image diagrams of FIGS. 9A, 9B, 9C and 9D after image alignment in accordance with the present invention.

FIGS. 9I, 9J, 9K and 9L are schematic diagrams respectively showing image diagrams of FIGS. 9E, 9F, 9G and 9H after being colored (visualizing electrodes) in accordance with the present invention.

FIG. 9M is a schematic enlarged diagram of FIG. 9I in accordance with the present invention.

FIG. 9N is a schematic diagram of FIG. 9M showing visualized electrodes in accordance with the present invention.

FIG. 10 is a schematic diagram showing integrated cross-sectional images of the brain, (intracranial) electrodes and blood vessels in accordance with the present invention.

FIG. 11 is a schematic perspective diagram showing integrated 3D images of the brain and blood vessels in accordance with the present invention.

FIG. 12 is a schematic perspective diagram showing integrated 3D images of the brain, (intracranial) electrodes and blood vessels in accordance with the present invention.

### DETAILED DESCRIPTION OF PREFERRED EMBODIMENT

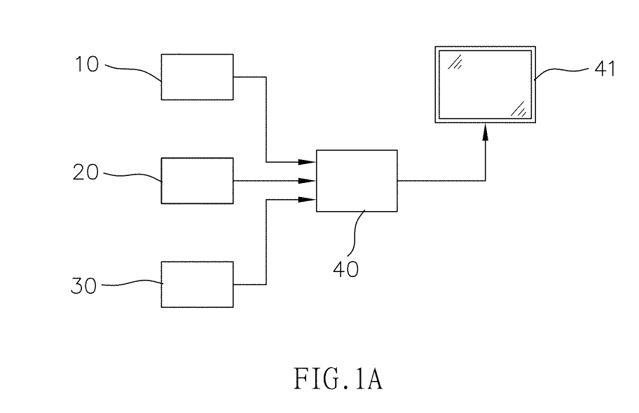

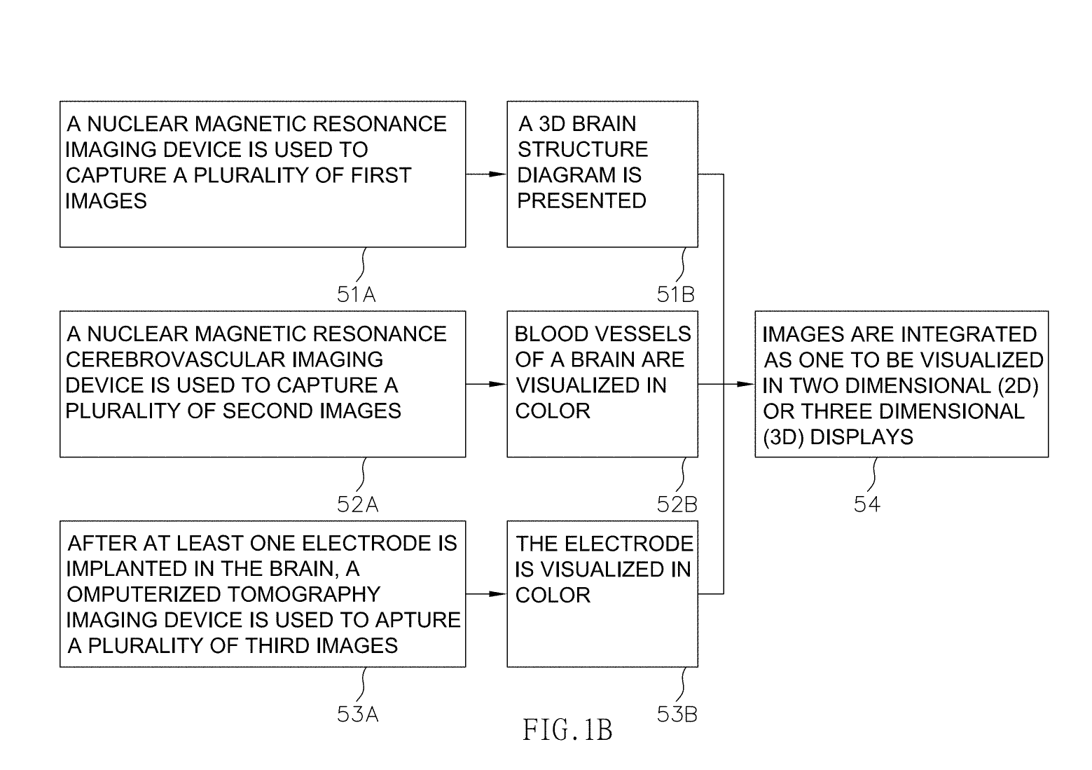

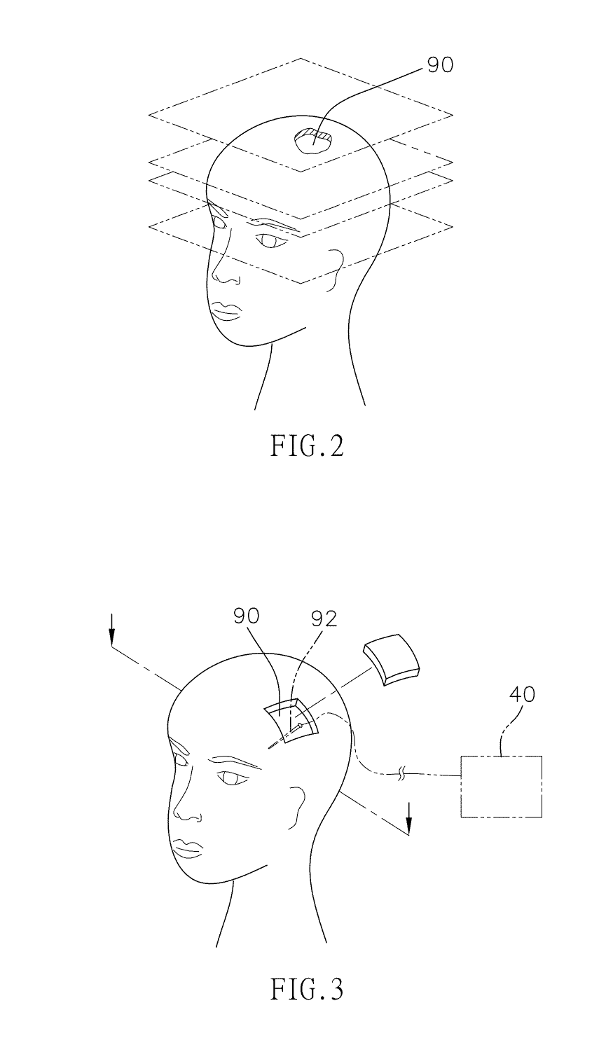

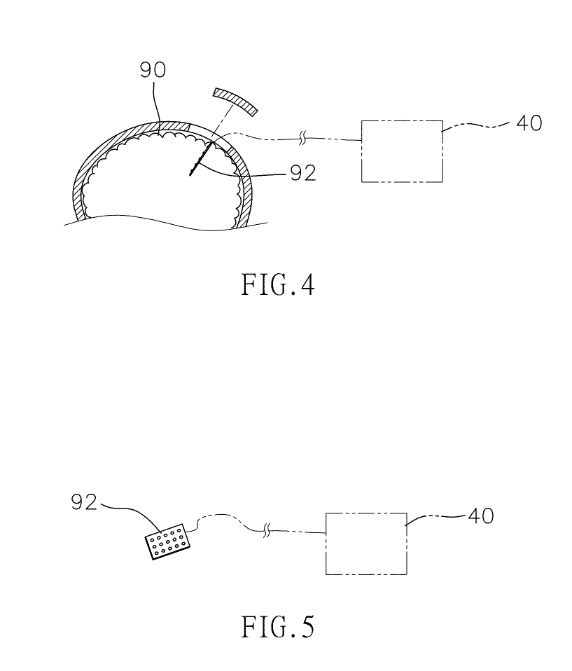

Referring to FIGS. 1A, 1B, 2, 3, 4 and 5, the present invention is related to an image simulation system and method for covisualizing electrodes and cortical vessels on a three dimensional (3D) brain structure diagram. The image simulation system comprises a first image capturing device 10, a second image capturing device 20, a third image capturing device 30, and a processing device 40.

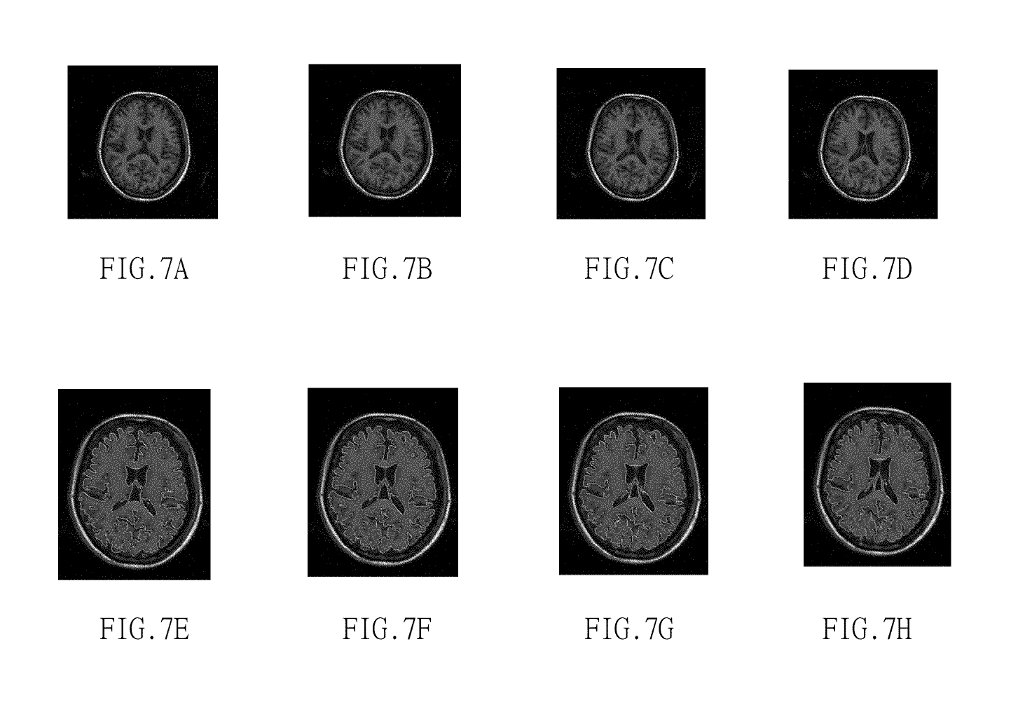

The first image capturing device 10 is used to capture a plurality of first images (for example, the 146th to 149th layers, referring to FIGS. 7A, 7B, 7C and 7D) of a brain 90 in advance. The plurality of first images are completely used to display a three-dimensional structure of the brain 90.

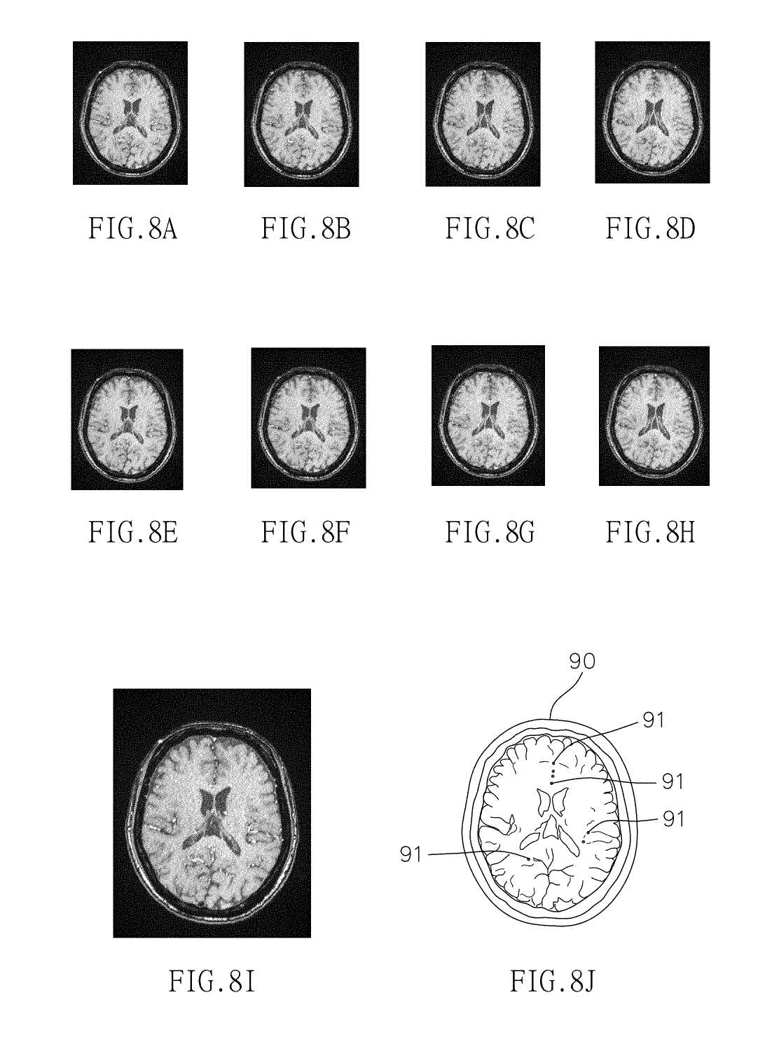

The second image capturing device 20 is used to capture a plurality of second images (for example, the 146th to 149th layers, referring to FIGS. 8A, 8B, 8C and 8D) of the brain 90 in advance. The plurality of second images are completely used to display blood vessels 91 of the brain 90 (referring to FIG. 8J, in fact, blood vessels 91 can be “cortical vessels”).

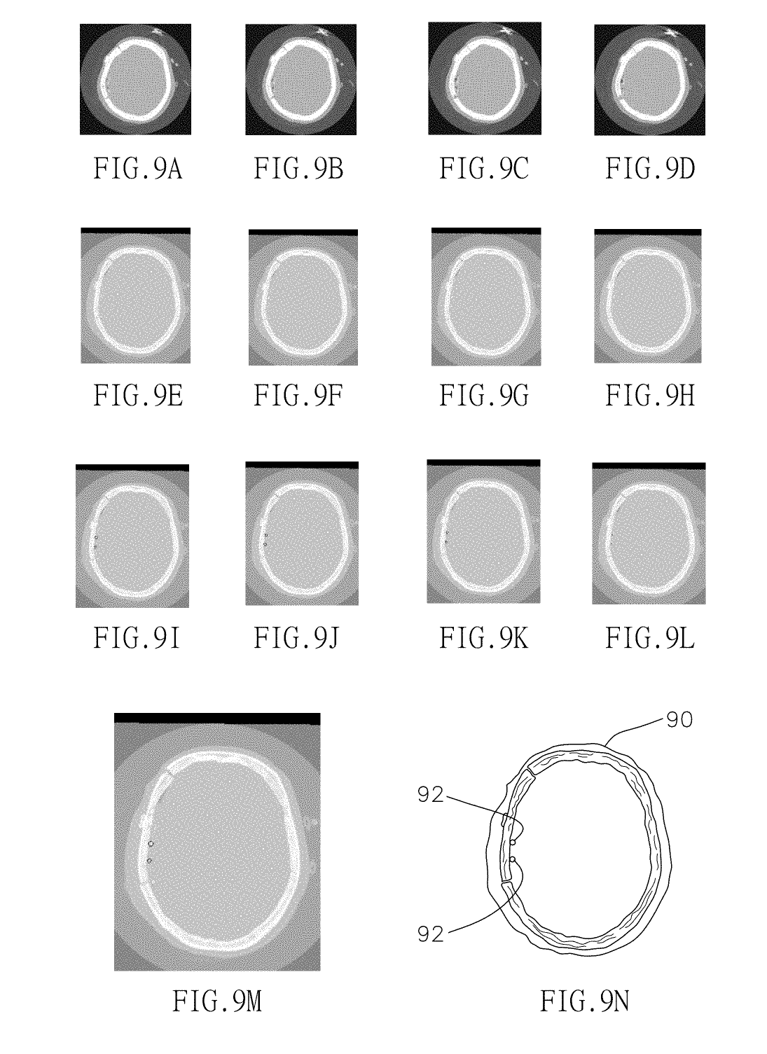

The third image capturing device 30 is used to capture a plurality of third images (for example, the 146th to 149th layers, referring to FIGS. 9A, 9B, 9C and 9D) of the brain 90 after the brain 90 is implanted with at least one electrode 92. The plurality of third images are completely used to display the at least one electrode 92 in the brain 90.

The processing device 40 is data-communicably connected to the first image capturing device 10, the second image capturing device 20 and the third image capturing device 30. The plurality of second images (referring to FIGS. 8A, 8B, 8C and 8D) are firstly retrieved and set as standards, and a plurality of first images (referring to FIGS. 7A, 7B, 7C and 7D) are then correspondingly retrieved and aligned with the plurality of retrieved second images in order to generate a plurality of aligned first images (referring to FIGS. 7E, 7F, 7G and 7H). Contrast between the blood vessel 91 and its adjacent non-vessel portions shown in the plurality of retrieved second images is firstly enhanced via image processing in order to visualize the blood vessel 91. The blood vessel 91 is further colored with a first color (such as red) in order to visualize a location of the blood vessel 91 at the brain 90 in the plurality of retrieved second images (referring to FIGS. 8E, 8F, 8G and 8H). In addition, the plurality of retrieved second images (referring to FIGS. 8A, 8B, 8C and 8D) are set as standards, and a plurality of third images (referring to FIGS. 9A, 9B, 9C and 9D) are correspondingly retrieved and aligned with the plurality of retrieved second images in order to generate a plurality of aligned third images (referring to FIGS. 9E, 9F, 9G and 9H). Contrast between the electrode 92 and its adjacent non-electrode portions shown in the plurality of aligned third images are firstly enhanced via image processing in order to visualize the electrode 92. The electrode 92 is further colored with a second color (such as blue) in order to visualize a location of the electrode 92 at the brain 90 in the plurality of aligned third images. Finally, the plurality of aligned first images, the plurality of colored second images and the plurality of colored third images are integrated to obtain integrated viewable information of a brain, (intracranial) electrodes and blood vessels for medical reference.

In practice, the first image capturing device 10 can be a nuclear magnetic resonance imaging device, for example, a nuclear magnetic resonance imaging device using three-dimension (3D) magnetization-prepared rapid acquisition gradient-echo (abbreviated as MP-RAGE or MP RAGE) sequence, or a high-resolution nuclear magnetic resonance imaging (abbreviated as MRI) device having the same function.

The second image capturing device 20 can be a nuclear magnetic resonance cerebrovascular imaging device, for example, a nuclear magnetic resonance cerebrovascular imaging device using time-of-flight (TOF) magnetic resonance angiography (abbreviated as TOF MRA) technology, or a high-resolution nuclear magnetic resonance imaging (abbreviated as MRI) device having the same function.

The third image capturing device 30 can be a computerized tomography (briefly referred as CT) imaging device, or any device with equivalent function.

It is required to particularly demonstrate that “nuclear magnetic resonance imaging device” and “nuclear magnetic resonance cerebrovascular imaging device” are both nuclear magnetic resonance imaging machines for image capturing or obtaining. They are different in image capturing sequences so as to result in imaging of different tissues and contrast difference.

The plurality of first images (for example, the 146th to 149th layers) are MRI images before the electrode 92 is implanted.

The plurality of second images (for example, the 146th to 149th layers) are MRI cerebrovascular images (Referring to FIGS. 8I and 8J) before the electrode 92 is implanted.

The plurality of third images (for example, the 146th to 149th layers) are computerized tomography images (Referring to FIGS. 9M and 9N) after the electrode 92 is implanted.

The foregoing exemplified embodiment of the present invention is only illustrated by describing the 146th to 149th layers for simplification. Layers of other sections or even all layers of the entire brain 90 of a head can be adopted to proceed.

The processing device 40 further comprises a display 41 used to display the integrated viewable information of the brain, (intracranial) electrodes and blood vessels.

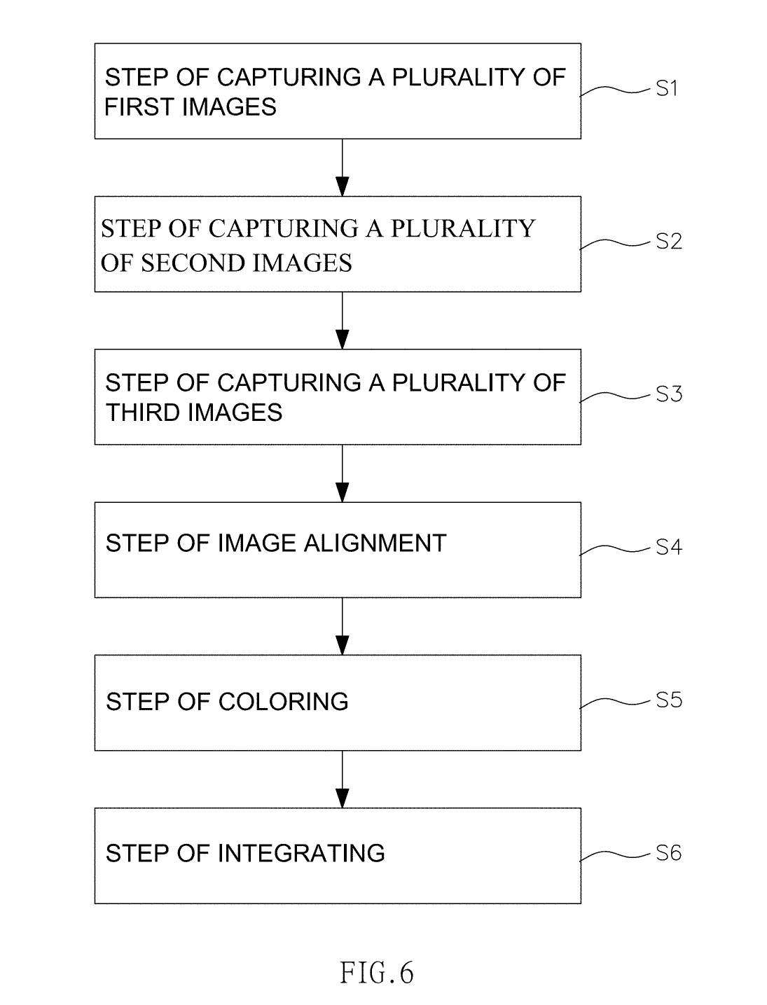

Referring to FIG. 6, an image simulation method in accordance with the present invention comprises the following steps.

1. A step S1 of capturing a plurality of first Images: The step S1 is proceeded by installing a first image capturing device 10 to capture a plurality of first images (for example, the 146th to 149th layers, referring to FIGS. 7A, 7B, 7C and 7D) of a brain 90 in advance. The plurality of first images are completely used to display a three-dimensional structure of the brain 90.

2. A step S2 of capturing a plurality of second Images: The step S2 is proceeded by installing a second image capturing device 20 to capture a plurality of second images (for example, the 146th to 149th layers, referring to FIGS. 8A, 8B, 8C and 8D) of the brain 90 in advance. The plurality of second images are completely used to display blood vessels 91 of the brain 90.

3. A step S3 of capturing a plurality of third Images: The step S3 is proceeded by installing a third image capturing device 30 to capture a plurality of third images (for example, the 146th to 149th layers, referring to FIGS. 9A, 9B, 9C and 9D) of the brain 90 after the brain 90 is implanted with at least one electrode 92. The plurality of third images are completely used to display the at least one electrode 92 in the brain 90.

4. A step S4 of image alignment: The step S4 is proceeded by retrieving the plurality of second images (referring to FIGS. 8A, 8B, 8C and 8D) as standards, and retrieving correspondingly a plurality of first images (referring to FIGS. 7A, 7B, 7C and 7D) for image adjustment and alignment so as to generate a plurality of aligned first images (referring to FIGS. 7E, 7F, 7G and 7H) by respectively matching a head contour in the plurality of second images. In the meantime, the step S4 is simultaneously proceeded by retrieving the plurality of second images (referring to FIGS. 8A, 8B, 8C and 8D) as standards, and retrieving correspondingly a plurality of third images (referring to FIGS. 9A, 9B, 9C and 9D) for image adjustment and alignment so as to generate a plurality of aligned third images (referring to FIGS. 9E, 9F, 9G and 9H) by respectively matching a head contour in the plurality of second images.

5. A step S5 of coloring: The step S5 is proceeded by enhancing contrast between the blood vessel 91 and its adjacent non-vessel portions shown in the plurality of retrieved second images via image processing to visualize the blood vessel 91, and followed by coloring the blood vessel 91 with a first color to visualize a location of the blood vessel 91 at the brain 90 in the plurality of retrieved second images (referring to FIGS. 8E, 8F, 8G and 8H). In the meantime, the step S5 is simultaneously proceeded by enhancing contrast between the electrode 92 and its adjacent non-electrode portions shown in the plurality of aligned third images via image processing to visualize the electrode 92 and followed by coloring the electrode 92 with a second color to visualize a location of the electrode 92 at the brain 90 in the plurality of aligned third images (referring to FIGS. 9I, 9J, 9K and 9L).

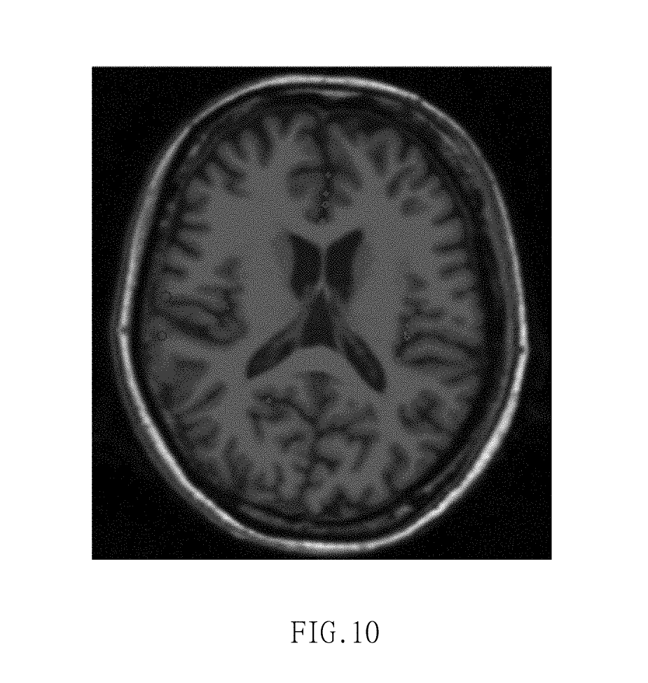

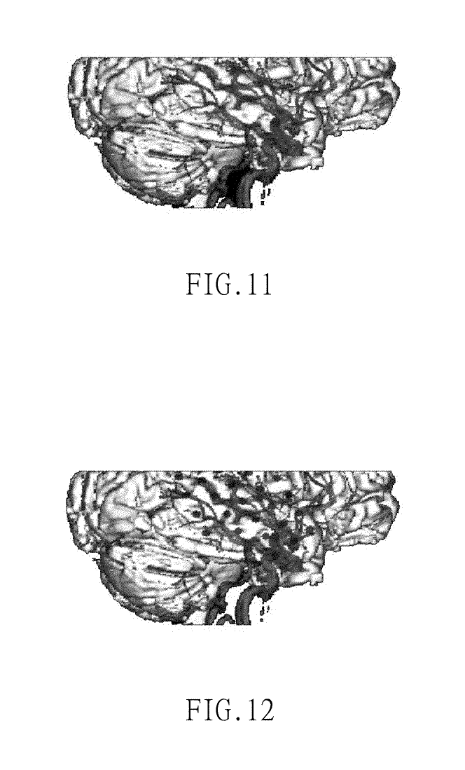

6. A step S6 of integrating: The step S6 is proceeded by integrating the plurality of aligned first images, the plurality of colored second images and the plurality of colored third images to obtain integrated viewable information (referring to FIGS. 10, 11 and 12) of a brain, (intracranial) electrodes and blood vessels for medical reference.

In practice, the first image capturing device 10 can be a nuclear magnetic resonance imaging device, for example, a nuclear magnetic resonance imaging device using three-dimension (3D) magnetization-prepared rapid acquisition gradient-echo (abbreviated as MP-RAGE or MP RAGE) sequence, or a high-resolution nuclear magnetic resonance imaging (abbreviated as MRI) device having the same function.

The second image capturing device 20 can be a nuclear magnetic resonance cerebrovascular imaging device, for example, a nuclear magnetic resonance cerebrovascular imaging device using time-of-flight (TOF) magnetic resonance angiography (abbreviated as TOF MRA) technology, or a high-resolution nuclear magnetic resonance imaging (abbreviated as MRI) device having the same function.

The third image capturing device 30 can be a computerized tomography (briefly referred as CT) imaging device, or any device with equivalent function.

It is required to particularly demonstrate that “nuclear magnetic resonance imaging device” and “nuclear magnetic resonance cerebrovascular imaging device” are both nuclear magnetic resonance imaging machines for image capturing or obtaining. They are different in image capturing sequences so as to result in imaging of different tissues and contrast difference.

The plurality of first images (for example, the 146th to 149th layers) are MRI images before the electrode 92 is implanted.

The plurality of second images (for example, the 146th to 149th layers) are MRI cerebrovascular images (Referring to FIGS. 8I and 8J) before the electrode 92 is implanted.

The plurality of third images (for example, the 146th to 149th layers) are computerized tomography images (Referring to FIGS. 9M and 9N) after the electrode 92 is implanted.

The foregoing exemplified embodiment of the present invention is only illustrated by describing the 146th to 149th layers for simplification. Layers of other sections or even all layers of the entire brain 90 of a head can be adopted to proceed.

The processing device 40 further comprises a display 41 used to display the integrated viewable information of the brain, (intracranial) electrodes and blood vessels.

Referring to FIG. 1B, an image simulation method in accordance with the present invention comprises the following.

A first process includes:

(a) in a step S1A, a nuclear magnetic resonance imaging device is used to capture a plurality of first images.

(b) in a step S1B, a 3D brain structure diagram is presented.

A second process includes:

(c) in a step S2A, a nuclear magnetic resonance cerebrovascular imaging device is used to capture a plurality of second images.

(d) in a step S2B, blood vessels 91 of a brain 90 are visualized in color.

A third process includes:

(e) in a step S3A, after at least one electrode 92 is implanted in the brain 90, a computerized tomography imaging device is used to capture a plurality of third images.

(f) in a step S3B, the electrode 92 is visualized in color.

Finally, in a step S4, the above mentioned images are integrated as one to be visualized in two dimensional (2D) or three dimensional (3D) displays.

Advantages and benefits of the present invention are depicted as follows.

(1) Spatial positions of implanted electrodes and blood vessels of brains can be confirmed in advance at a time of medical surgery. Through integrated viewable information of a brain, (intracranial) electrodes and blood vessels generated from the present invention, physicians can physically understand the spatial positions of the implanted electrodes and blood vessels of brains during medical surgery. Therefore, space and positions of implanted electrodes and blood vessels of brains can be confirmed in advance at the time of medical surgery.

(2) Physicians can avoid bleeding due to accidental touch on blood vessels. When actual distribution of blood vessels of brains is well known in advance, physicians can avoid inadvertent contact on larger blood vessels to further cause tremendous bleeding during intracranial surgery. Therefore, bleeding due to accidental touch on blood vessels can be successfully avoided.

The above disclosure of preferred embodiment of the present invention is only used to describe and explain the present invention in details. Simple modifications and changes made to the preferred embodiment are deemed to be covered by the following claims of the present invention without departing from spirit and scope of the present invention.

What is claimed is:

1. An image simulation system to covisualize electrodes and cortical vessels on three dimensional (3D) brain structure diagram, comprising:
a first image capturing device used to capture a plurality of first images of a brain in advance, wherein the plurality of first images are completely used to display a three-dimensional structure of the brain;
a second image capturing device used to capture a plurality of second images of the brain in advance, wherein the plurality of second images are completely used to display blood vessels of the brain;
a third image capturing device used to capture a plurality of third images of the brain after the brain is implanted with at least one electrode, wherein the plurality of third images are completely used to display the at least one electrode in the brain; and
a processing device being data-communicably connected to the first image capturing device, the second image capturing device and the third image capturing device, wherein the plurality of second images are firstly retrieved by the processing device and set as standards, and a corresponding plurality of first images are then retrieved and aligned with the plurality of retrieved second images in order to generate a plurality of aligned first images, contrast between each of the blood vessels and its adjacent non-vessel portions shown in the plurality of retrieved second images is firstly enhanced via image processing of the processing device in order to visualize the each blood vessel, the each blood vessel is further colored with a first color in order to visualize a location of the each blood vessel at the brain in the plurality of retrieved second images, the plurality of retrieved second images are set as standards, and a corresponding plurality of third images are retrieved and aligned with the plurality of retrieved second images in order to generate a plurality of aligned third images, contrast between the at least one electrode and its adjacent non-electrode portions shown in the plurality of aligned third images are firstly enhanced via image processing of the processing device in order to visualize the at least one electrode, the at least one electrode is further colored with a second color in order to visualize a location of the at least one electrode at the brain in the plurality of aligned third images, the plurality of aligned first images, the plurality of colored second images and the plurality of colored third images are integrated to obtain integrated viewable information of the brain, the at least one electrode and the blood vessels for medical reference.

2. The image simulation system to covisualize electrodes and cortical vessels on 3D brain structure diagram as claimed in claim 1, wherein:
the first image capturing device is a nuclear magnetic resonance imaging device;
the second image capturing device is a nuclear magnetic resonance cerebrovascular imaging device; and
the third image capturing device is a computerized tomography imaging device.

3. The image simulation system to covisualize electrodes and cortical vessels on 3D brain structure diagram as claimed in claim 1, wherein the plurality of first images are nuclear magnetic resonance imaging (MRI) images before the at least one electrode is implanted.

4. The image simulation system to covisualize electrodes and cortical vessels on 3D brain structure diagram as claimed in claim 1, wherein the plurality of second images are nuclear magnetic resonance imaging (MRI) cerebrovascular images before the at least one electrode is implanted.

5. The image simulation system to covisualize electrodes and cortical vessels on 3D brain structure diagram as claimed in claim 1, wherein the plurality of third images are computerized tomography images after the at least one electrode is implanted.

6. The image simulation system to covisualize electrodes and cortical vessels on 3D brain structure diagram as claimed in claim 1, wherein the processing device further comprises a display used to display the integrated viewable information of the brain, the at least one electrode and the blood vessels.

7. An image simulation method to covisualize electrodes and cortical vessels on three dimensional (3D) brain structure diagram, comprising:
installing a first image capturing device in a step of capturing a plurality of first Images to capture a plurality of first images of a brain in advance, wherein the plurality of first images are completely used to display a three-dimensional structure of the brain;
installing a second image capturing device in a step of capturing a plurality of second Images to capture a plurality of second images of the brain in advance, wherein the plurality of second images are completely used to display blood vessels of the brain;
installing a third image capturing device in a step of capturing a plurality of third Images to capture a plurality of third images of the brain after the brain is implanted with at least one electrode, wherein the plurality of third images are completely used to display the at least one electrode in the brain;
retrieving the plurality of second images as standards in a step of image alignment, and retrieving correspondingly a plurality of first images for image adjustment and alignment so as to generate a plurality of aligned first images via respectively matching a head contour in the plurality of second images; simultaneously retrieving the plurality of second images as standards, and retrieving correspondingly a plurality of third images for image adjustment and alignment so as to generate a plurality of aligned third images via respectively matching the head contour in the plurality of second images;
enhancing contrast between each of the blood vessels and its adjacent non-vessel portions shown in the plurality of retrieved second images via image processing in a step of coloring to visualize the each blood vessel, and coloring the each blood vessel with a first color to visualize a location of the each blood vessel at the brain in the plurality of retrieved second images; simultaneously enhancing contrast between the at least one electrode and its adjacent non-electrode portions shown in the plurality of aligned third images via image processing to visualize the at least one electrode, and coloring the at least one electrode with a second color to visualize a location of the at least one electrode at the brain in the plurality of aligned third images; and
integrating the plurality of aligned first images, the plurality of colored second images and the plurality of colored third images in a step of integrating to obtain integrated viewable information of the brain, the at least one electrode and the blood vessels for medical reference.

8. The image simulation method to covisualize electrodes and cortical vessels on 3D brain structure diagram as claimed in claim 7, wherein:
the first image capturing device is a nuclear magnetic resonance imaging device;
the plurality of first images are nuclear magnetic resonance imaging (MRI) images before the at least one electrode is implanted;
the second image capturing device is a nuclear magnetic resonance cerebrovascular imaging device;
the plurality of second images are nuclear magnetic resonance imaging (MRI) cerebrovascular images before the at least one electrode is implanted;
the third image capturing device is a computerized tomography imaging device; and
the plurality of third images are computerized tomography images after the at least one electrode is implanted.

9. The image simulation method to covisualize electrodes and cortical vessels on 3D brain structure diagram as claimed in claim 7, wherein the processing device further comprises a display used to display the integrated viewable information of the brain, the at least one electrode and the blood vessels.
